(19) United States
(12) Patent Application Publication
(10) Pub. No.: US 2009/0216082 A1
(43) Pub. Date: Aug. 27, 2009

(54) Device, System and Method for In Vivo Magnetic Immunoassay Analysis

(76) Inventor: Elisha Rabinovitz, Haifa (IL)

Correspondence Address: Pearl Cohen Zedek Latzer, LLP, 1500 Broadway, 12th Floor, New York, NY, 10036, US

(21) Appl. No.: 11/887,640

(22) PCT Filed: Apr. 2, 2006
(86) PCT No.: PCT/IL2006/000419

§ 371 Date: Mar. 11, 2009

Related U.S. Application Data
(60) Provisional application No. 60/667,097, filed on Apr. 1, 2005.

(51) Int. Cl. A61B 1/00

(52) U.S. Cl. 600/118

(57) ABSTRACT

A device system, and method may provide in-vivo detection of target molecules in an endo-luminal sample, for example for the detection of cancer in the gastrointestinal tract, utilizing for example an in-vivo sensing device. the sensing device may accept samples of fluids from a body lumen and detect the contents of that sample for example by introducing paramagnetic particles to the sample of fluids, immobilizing a target molecule bonded to a paramagnetic particle to a reaction channel, and detecting bonded paramagnetic particles.

### FIELD OF THE INVENTION

The present invention relates to in vivo analysis in general and to analysis using swallowable devices in particular.

### BACKGROUND OF THE INVENTION

An atypical concentration or presence of substances in body fluids or in body lumens may be indicative of the biological condition of the body. For example, the presence of elevated concentrations of red blood cells in the gastrointestinal (GI) tract may indicate different pathologies, depending on the location of the bleeding along the GI tract. Likewise, abnormalities in physical conditions of the body, such as elevated temperature, may indicate pathology. Early detection, identification and location of abnormal conditions are critical for correctly diagnosing and treating various pathologies.

Medical detection kits are usually based on in vitro testing of body fluid samples for the presence of a suspected substance. This method of detection does not easily enable the localization or identification of the origin of an abnormally occurring substance. In many instances localizing an abnormally occurring substance in a body lumen greatly contributes to the identification of pathology, and thus contributes to the facile treatment of the identified pathology. For example, bleeding in the stomach may indicate an ulcer while bleeding in the small intestine may indicate the presence of a tumor.

In some cases, diseases, such as cancer, are detected by analyzing the blood stream for tumor specific markers, typically, specific antibodies. One of the drawbacks of this method is that the appearance of antibodies in the blood stream usually occurs at a late stage of the disease, such that early detection is not possible by this method.

The detection of pathologies in the GI tract is possible by endoscopy, however this possibility is limited to the upper or lower gastrointestinal tract. Thus, pathologies in other parts of the GI tract, such as the small intestine, may not be easily detected by Endoscopy.

### SUMMARY OF THE INVENTION

According to embodiments of the present invention an in-vivo device for in-vivo magnetic immunoassay analysis may include a reaction channel to accept a fluid sample in-vivo, paramagnetic particles conjugated with a receptor specific to a target molecule that may be present within the in-vivo fluid sample, a magnetic sensor to detect the paramagnetic particles bonded with the target molecule; and a magnet.

According to one embodiment of the present invention, the bonded paramagnetic particles may be immobilized onto the reaction channel. In one example, an alternating magnetic field may be applied and a magnetic sensor may detect changes in the magnetic field due to the immobilized paramagnetic particles. Detected changes may be transmitted to an external source for further analysis and for display.

According to an embodiment of the present invention the in-vivo device may be capsule, e.g. a swallowable capsule.

According to another embodiment of the present invention, a method for in vivo magnetic immunoassay analysis may include introducing paramagnetic particles to the fluid sample within an in-vivo device, wherein the paramagnetic particles may be conjugated with a receptor specific to a target molecule, applying a magnetic field in the vicinity of the paramagnetic particles, and detecting the paramagnetic particles that bonded to the target molecules.

According to yet another embodiment of the present invention, a system for in-vivo magnetic immunoassay analysis may include a sensing unit to magnetically detect the presence of a target molecule within a fluid sample, a transmitter for wirelessly transmitting an output from the sensing unit, an external receiver for wirelessly receiving the output from the sensing unit, and a processor for processing the output from the sensing unit.

According to one embodiment of the present invention the sensing unit may be fully incorporated within an in-vivo device, e.g. a swallowable capsule.

### BRIEF DESCRIPTION OF THE DRAWINGS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

FIG. 1 is a schematic, longitudinal cross-section illustration of an in vivo device, constructed and operative in accordance with an embodiment of the present invention;

FIG. 2 is a schematic longitudinal cross-section illustration of a sensing unit of an in-vivo device according to one embodiment of the present invention;

FIG. 3 is a schematic illustration of a reaction channel to be incorporated within an in-vivo device according to an embodiment of the present invention;

FIG. 4 is a simplified flow chart describing a method for in-vivo detection of target molecules according to embodiments of the present invention;

FIGS. 5A-5C is a schematic illustration of a pump-mixer that may be integrated with a silicon chip according an embodiment of the present invention; and

FIG. 6 is a schematic illustration of an in-vivo sensing system according to embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

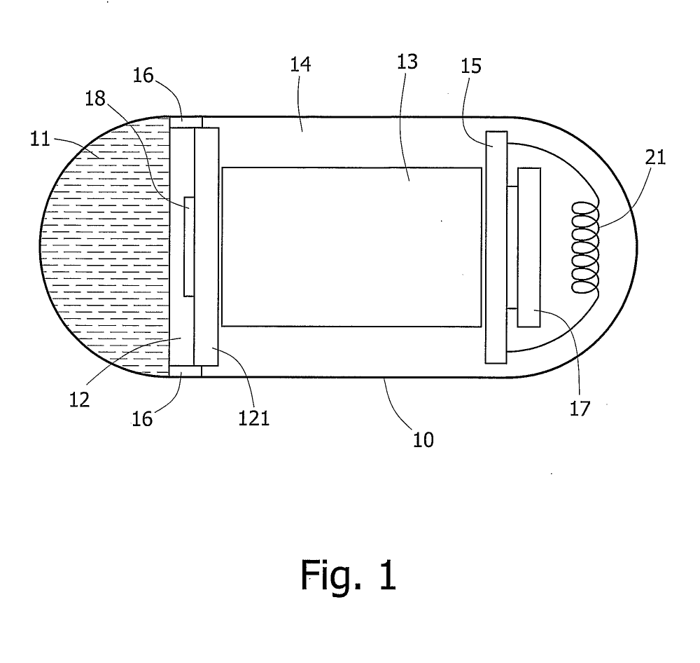

Reference is now made to FIG. 1, which depicts an in-vivo device, such as a swallowable capsule 10, which may, according to one embodiment, comprise a reaction chamber and/or reaction channel 12, a magnetic sensor, for example a silicon chip 121 including sensing traces, and at least one reagent reservoir 14, containing and/or storing a suspension of paramagnetic beads and/or particles conjugated with an antibody, to react with a specific target molecule and/or antigen if present, in an endo-luminal body fluid analyte sample. In some examples, magnetic particles and/or beads may be used. Reaction channel 12 may include receptors, e.g. antibodies trapped in reaction channel 12 to react with target molecules in an endo-luminal body fluid analyte sample. Paramagnetic particles conjugated with a receptor specific to a target molecule may be introduced to the fluid sample.

The use of magnetic biosensors to detect antigens may be known in the art. Multi-sensing chips capable of detecting a range of different agents using magnetic beads may be described by Rife at al. (US 20040253744).

According to another embodiment capsule 10 and/or an alternate in-vivo device may also comprise additional reservoirs with additional reagents such as buffers to adjust pH or reagents to reduce non specific adsorption. According to one embodiment at least one analyte inlet 18 may lead to the reaction channel 12 containing one or more specific receptors (e.g. an antibody) capable of immobilizing and/or capturing the desire target molecule (e.g. an antigen) while the rest of the unbounded analyte may be absorbed in garbage reservoir and/or pad 11. After a predetermined time (e.g., after enough analyte passed into the sensing compartment) conjugated beads from reservoir 14 may be pumped into the reaction channel 12 and may attach and/or bond to the captured molecules (e.g. the previously captured antigen). A magnetic field may be introduced by a magnet 13, e.g. electromagnet, in the vicinity of the reaction channel 12 and/or the paramagnetic particles within the reaction channel 12. The amount and/or concentration of paramagnetic beads may be measured with the aid of the silicon chip 121 including sensing traces that may measure disturbances in the magnetic field due to the presence of the paramagnetic beads and/or particles trapped and/or bonded within the reaction channel 12. The results and/or output from the sensing traces may be transmitted outside the body lumen via a transmitter located, for example, on PCB board 15 that may also include, for example, a controller for controlling operations of the capsule 10. Antenna 21 may transmit data, by wireless communication to an external source. The power to operate the system may for example, be supplied by battery 17, or may be received from an external source by, for example, power induction.

According to an embodiment of the invention the device may include a coating 16 to cover for example sample inlet 18 made, for example, of pH sensitive material that may dissolve in a specified organ to be tested. e.g. a coating that dissolves at pH 5 or more will not be dissolved in the stomach but will be dissolved once the capsule has reached the small intestine. Thus only secretions and/or endo-luminal fluids from the intestine may be measured but not secretions from the stomach. In another embodiment the analyte may enter through other types of gates that may be for example controlled by a controller included within the capsule and/or by commands received from an external receiving device.

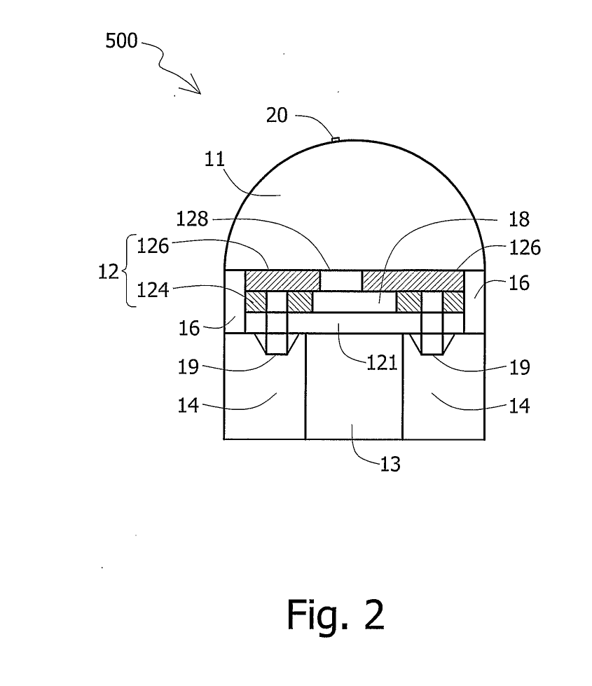

Reference is now made to FIG. 2, showing a more detailed schematic of the sensing unit 500 of sensing device 10, according to an embodiment of the present invention. Sensing unit 500, may include one or more reaction channels 12 including an inlet 18 through which endo-luminal bodily fluid sample may enter, one or more reagent reservoirs 14 including reservoir openings or valves 19 allowing contents from the reservoir 14 to enter reaction channel 12, and a waste chamber 11 including a waste chamber inlet 128 that may allow excess fluid in reaction chamber 12 to be evacuate into the waste chamber 11.

According to embodiments of the present invention, at least one of reagent reservoirs 14 may contain a suspension of paramagnetic beads and/or particles conjugated with an antibody, to react with a specific antigen if present, in an endo-luminal body fluid analyte sample. Other reagent reservoirs 14 may contain additional reagents, for example, buffers to adjust pH or reagents to reduce non specific adsorption. Opening valve 19 may be any suitable opening or valve for dispensing the contents of reagent reservoirs 14 at a required time and/or on demand.

Waste chamber 11 may include an absorbing pad that may absorb excess fluid sample from reaction channel 12 through opening 128. In one example, excess air trapped in the pad during absorption may be released through an opening 20.

Magnet 13 may induce the reaction channel 12 with a desired magnetic field, for example, after the paramagnetic beads conjugated with an antibody, may react with a specific antigen if present, in reaction channel 12.

Silicon chip 121, may sense local changes in the magnetic field, for example due to the presence of paramagnetic particles present in the reaction channel 12 after, for example, reacting with a specific antigen if present, in an endo-luminal body fluid analyte sample.

According to one embodiment of the present invention, reaction chamber 12 may include two layers. In one example, a first layer 124 may be made of glass, silica, plastic or any other suitable material. One or more channels may be etched on to first layer 124. One or more specific receptors, e.g. antibodies may be immobilized on sites throughout the channels formed on first layer 124 using known methods. In one example, receptors specific to a single target molecule may be immobilized, for example by coating, onto different sections of first layer 124. In another example, different types of receptors may be immobilized onto first layer 124 to allow simultaneous detection of more than one type of target molecule. A second layer 126 may cover 124 to seal, at least partially, the channels formed in first layer 124. Second layer 126 may be adhered to first layer 124. In one embodiment the sample of body fluid analyte can enter sensing compartment 12 through opening 18 with the aid of a pump e.g. piezoelectric pump, or any other on-chip pump. Micro-pumps on chip are known in the art, such as NEC's thin profile (5 mm tick) high pressure piezoelectric pump on chip. In another embodiment the flow through reaction channels 12 may be designed to use capillary forces that drive the sample from opening 18 through reaction channel 12 and opening 128 into a waste chamber 11 and/or garbage zone equipped with an absorbing pad with no electromechanical pumps.

Silicon chip 121 may be a third layer positioned below first layer 124. Silicon layer 124 may include a distribution of sensing tracer that may sense changes in a magnetic field, for example, due to the presence of paramagnetic particles immobilized onto reaction channel 12. According to embodiments of the present invention, the sensing tracers may be Hall Effect based sensors, or Giant MagnetoResistors (GMR) that may, for example, measure a change of resistance due to a change in the magnetic field. The sensing tracers may be positioned on the silicon chip 121 in positions that correspond to the position of each and/or a group of receptors immobilized onto first layer 124 to measure the change in magnetic field due to the presence of the antigen bonded to the paramagnetic particles and immobilized onto a specific location in the reaction channel 12. Other methods of measuring the presence of paramagnetic particles immobilized onto a reaction channel may be implemented. The multiple sensing tracers may each be wired to pin-outs of the silicon chip 121. Additional sensing zones may be added for reference, control or other applications.

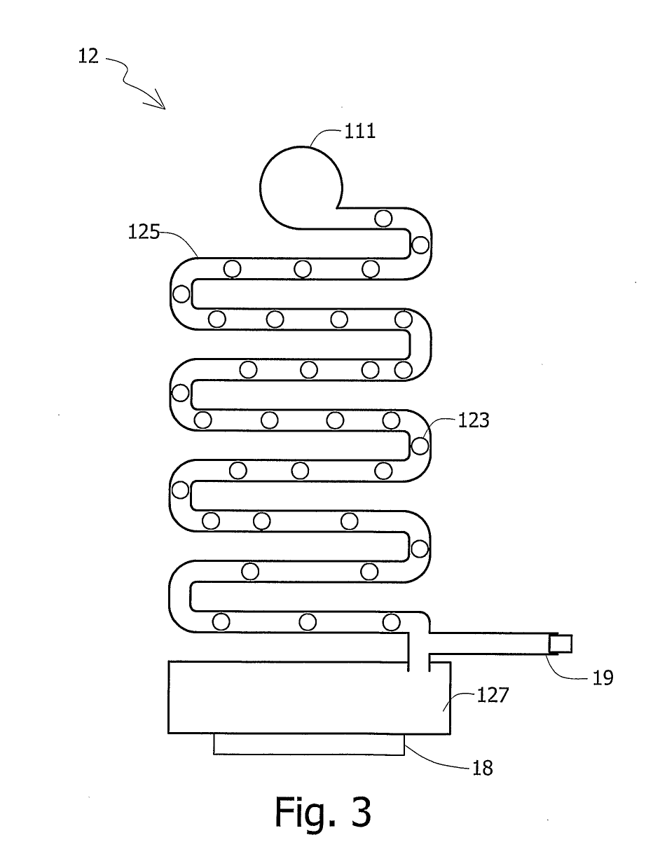

Reference is now made to FIG. 3 showing a more detailed schematic of the reaction channel 12 according to embodiments of the present invention. Reaction channel 12 may include one or more channels 125, e.g. etched onto first layer 124, coated with specific receptors 123 that may be immobilized onto specific regions of channels 125. Inlet and/or opening 18 may allow endo-luminal fluid to be pumped into channel 125 via a pump 127. Pump 127 may be, for example, a piezoelectric pump or any other pump mounted on and/or controlled by silicon chip 121. Inlet 19 may allow reagent, e.g. a buffer with a suspension of paramagnetic beads, from reagent reservoir 14 to enter into reaction channel 12. Inlet 19 may include pump, or any suitable mechanism to promote flow from reagent chamber to reaction channel 12 on demand. Outlet 111 from reaction channel 12 may be fluidically connected to waste chamber inlet 128 and may provide passage of excess endo-luminal fluid and/or reagent to pass into the waste chamber 11 as may be described herein.

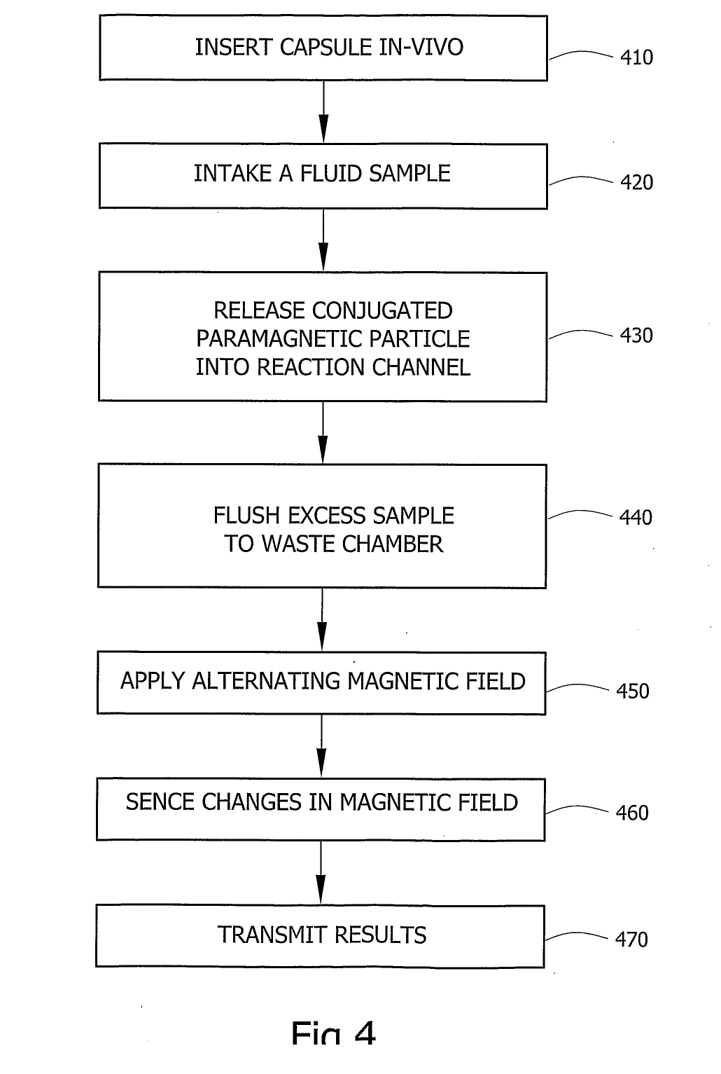

Reference is now made to FIG. 4 describing a method for detecting target molecules that may be present in endo-luminal fluids in-vivo according to an embodiment of the present invention. According to some embodiments, the capsule 10 and/or other in-vivo device may be positioned in-vivo or may pass through an organ and/or body lumen specified for investigation (e.g. stomach, small intestine, or colon) (block 410). A sample of body fluid may enter reaction channel 12 (block 420). As may be described herein, specific receptor molecules may have been previously coated and/or immobilized onto reaction channel 12. Target molecules that may be present in the sample of body fluids may be bonded to the receptor molecules. A suspension containing paramagnetic particles conjugated with a second set of receptor molecules from the same set of targets as the one immobilized onto the reaction channel 12 may be pumped through and/or released through opening 19 into the reaction channel 12 (block 430). While passing through reaction channel 12, part of the particles may bind to the target molecules already immobilized onto reaction channel 12. Excess sample fluid and unbounded paramagnetic particles may be flushed into waste chamber 11 (block 440). In one example, flushing may be facilitated by flow of the suspension containing the paramagnetic particles. In another example, the contents of an additional reagent reservoir 14, for example, a reagent reservoir containing a buffer may be released into reaction channel 12 to flush the reaction channel 12 from excess and/or unbounded paramagnetic particles. In yet another example, an additional sample of endo-luminal fluid may be used to flush the reaction channel 12 from excess and/or unbounded paramagnetic particles.

In one embodiment of the present invention, after passing the conjugated particles into reaction channel 12, magnet 13 may be operated to create a magnetic gradient that may pull off paramagnetic particles that may have non-specifically bonded to the surface of reaction channel 12 (block 450). Thus only beads with a specific bond to the target may remain bonded.

According to one embodiment of the present invention, the presence and the concentration of bonded paramagnetic particles, e.g. paramagnetic particles bonded to a target molecule, may be detected by exposing the bonded paramagnetic particles to an alternating magnetic field, e.g. an alternating magnetic field produced by magnet 13 (block 460). The alternating magnetic field may excite the bonded particles, resulting in local changes in the magnetic field. The change in the magnetic field may be proportional to the number of bounded particles such that the concentration of target molecules (e.g. antigens) may be established. An array of magnetic sensors, e.g. Hall Effect type sensors and/or GMR, embedded on a silicon chip 121 may sense local changes in the magnetic field at each of the sites of the bounded receptor molecules. Output from the magnetic sensor may be transmitted to an external source for further analysis and for report (block 470). Alternately, output from the magnetic sensor may be stored in a memory unit within the capsule and/or undergo processing before being transmitted to an external source.

In another embodiment of the present invention, nano-superparamagnetic particles may be used instead of the micro paramagnetic particles described herein. Nano-superparamagnetic may be very small with high mobility. As such they may be mixed with the sample stream to create the sandwich reaction in one step, enabling a continuous detection process.

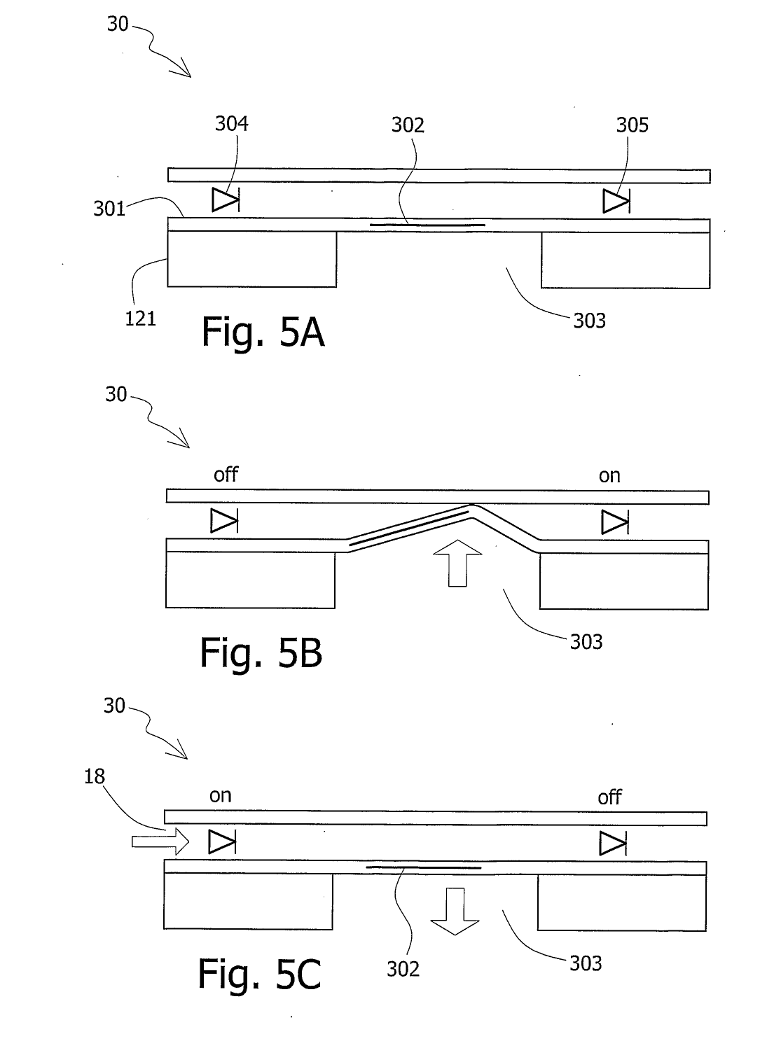

According to another embodiment of the present invention, a pump-mixer may be used and the magnet 13 may be used to operate the pump-mixer. Reference is now made to FIG. 5A-5C showing a cross section view of pump-mixer 30, e.g. a pump-mixer, in three situations. FIG. 5A schematically illustrates unit 30 in rest. Silicon chip 121 may be covered with a flexible membrane 301 adhered to the chip 121 on both sides of cavity 303. A paramagnetic piece 302 may be embedded or adhered to the membrane 301 in the vicinity of cavity 303. In one embodiment of the present invention, the channel 125 may be formed by membrane 301 together with cover chip 126 made of glass, silica, plastic or any other suitable material, leading from opening 18 to waste chamber 11. Two electromechanically operated valves 304 and 305, e.g. normally closed MEMS and/or step valves, may be located in channel 125 from both sides of cavity 303. Thus, at rest, the two valves may seal the reaction channel 12. Once the system is activated valve 304 may be activated enabling the analyte to enter through opening 18. In a subsequent step valve 304 may be closed, valve 305 may be activated, e.g. opened, and the magnet 13 (not shown in FIGS. 5A-C) may be activated. The magnetic field formed by the magnet may push the metal piece 302 upwards thus pushing the liquid forward into the reaction channel 12 (FIG. 5B). At yet another subsequent step, step valve 305 may be closed the magnetic field may be stopped or activated in the opposite direction and valve 304 may be opened. As a result the membrane 301 may be returned to its original or relaxed position and/or even pushed into cavity 303, by doing so new analyte may be sucked into the device through opening 18 (FIG. 5C). As such, multiple samples of endo-luminal fluids may enter reaction channel 12 and be tested for the presence of specified target molecules.

In yet another embodiment, sample may be introduced and mixed with a suspension of paramagnetic nanoparticles carrying appropriate antibodies that may be introduced through opening 19, into mixing-pumping device 30 (e.g. device described in FIGS. 5A-C). The analyte may be driven by mixing pump 30 through the reaction channel 12 including the immobilized receptors. The rest of the analyte may be released into waste chamber 12. Low frequency pulses of magnetic field may be applied in a plane parallel to the reaction channel 12. In the presence of this aligning field the nanoparticles may develop a net magnetization, which may relax when the field is turned off. Unbound nanoparticles relax rapidly by Brownian rotation and contribute no measurable signal. Nanoparticles that are bound to the target on the film may be immobilized and may undergo Néel relaxation, producing, for example, a slowly decaying magnetic flux, which may be detected by silicon chip 121. The ability to distinguish between bound and unbound labels may enable non-homogeneous assays, which do not require separation and removal of unbound paramagnetic particles. More over by using alternating magnetic fields the particles may be “vibrated” to improve mixing and better contact with the sensing plane.

In other embodiments reagent may be mixed by other possible methods or different micro-pump for different fluids may be used. For example, one micro-pump may be used for the in-vivo sampling of body fluids, another pump for the paramagnetic particles suspension, both pumping the fluids into a static on-chip mixer.

According to one embodiment, the capsule 10 may be a one time sample detector that may sample endo-luminal fluid from a specific site in a body lumen and detect the presence of one or more target molecules. According to another embodiment of the present invention, capsule 10 may be used for continuous sampling and detection and/or for sampling and detection at a specified frequency. For example, capsule 10 may sample endo-luminal every period of time, e.g. every half hour, and silicon chip 121 may take measurements after every sampling period. The results of the measurements may be cumulative, such that the detected changes for subsequent measurement will increase if more target molecules were detected. Consecutive sampling may be continued until all and/or most of the receptors immobilized onto the reaction channel are used up and/or occupied.

In yet another example, capsule 10 may include an array of sampling channels 12. Consecutive sampling may be achieved by activating a different and/or new sampling channel per measurements.

In one example of the present invention, all the receptor molecules may be specific to a single antigen and/or target molecule. In another example a range of receptor molecules may be used that may be specific to a range of different antigens may be used.

Embodiments of the present invention may be used in conjunction with an in-vivo sensing system or device such as described in US Application Publication Number US20020111544 to Iddan and published on Aug. 15, 2002 and entitled “System and Method for determining In-Vivo Body Conditions”, which is hereby incorporated by reference. The system according to other embodiments may be used in conjunction with an imaging capsule similar to embodiments described in U.S. Pat. No. 5,604,531 to Iddan et al. and/or U.S. Pat. No. 7,009,634 to Iddan et al. entitled “Device for In-Vivo Imaging”, all of which are hereby incorporated by reference.

Reference is now made to 6 showing a schematic illustration of an in-vivo sensing system according to embodiments of the present invention.

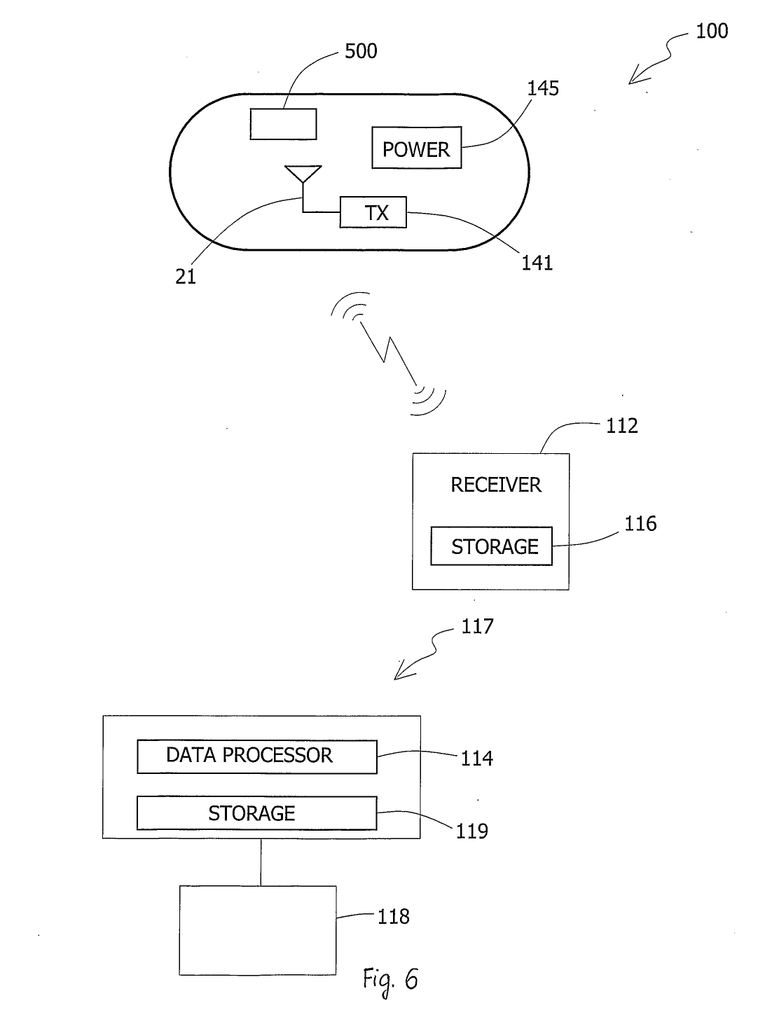

FIG. 6 is a schematic illustration of an in-vivo sensing system 100 in accordance with some embodiments of the invention. One or more components of system 100 may be used in conjunction with, or may be operatively associated with, the devices and/or components described herein or other in-vivo devices in accordance with embodiments of the invention.

In some embodiments, system 100 may include a device 10 having a sensing unit 500, e.g., a sensing unit to detect the presence of target molecules present in an in-vivo endo-luminal fluid sample, a power source 145, a transmitter 141, and an antenna 21. In some embodiments, device 10 may be implemented using a swallowable capsule, but other sorts of devices or suitable implementations may be used. Sensing unit 500 may magnetically detect the presence of a target molecule within a fluid sample and output from the sensing unit may be transmitted to an external receiving device.

Device 10 typically may be or may include an autonomous swallowable capsule, but device 10 may have other shapes and need not be swallowable and/or autonomous. Embodiments of device 10 are typically autonomous, and are typically self-contained. For example, device 10 may be a capsule or other unit where all the components are substantially contained within a container or shell or housing, and where device 10 does not require any wires or cables to, for example, receive power and/or transmit information. In some embodiments, device 10 may be autonomous and non-remote-controllable; in another embodiment, device 10 may be partially or entirely remote-controllable. In some examples, device 10 may include a receiver and receiving capability, e.g. to receive commands wirelessly from an external source.

Outside a patient's body may be, for example, an external receiver/recorder 112, which may include, or may be associated with, one or more antennas (or antenna elements), optionally arranged as an antenna array. Receiver/recorder 112 may receive signals transmitted by the in-vivo device 10, for example, signals carrying image data, sensed data, control data, or the like. Receiver/recorder 112 may, for example, store the received data in a memory unit or a storage unit 116. In some example, receiver/recorder 112 may include processing capability, user input capability and/or display capability. In some examples, receiver/recorder 112 may include a transmitter and transmitting antennas to transmit, e.g. by wireless connection, commands and data to in-vivo device 10.

Additionally, outside a patient's body may be, for example, a storage unit 119, a processor 114, and a monitor 18, which may optionally be implemented as a workstation 117, e.g., a computer or a computing platform. Workstation 117 may be connected to receiver/recorder 112 through a wireless or wired link or connection. Workstation 117 may receive from receiver/recorder 112 data that is received and/or recorded by receiver/recorder 112. In some embodiments, workstation 117 may receive the data from receiver/recorder 112 substantially in real-time, and/or while receiver/recorder 112 continues to receive and/or record data from the in-vivo device 10 and while the in-vivo device 10 is operational and/or in-vivo. In some embodiments, device 10 may communicate with the external receiving and display system (e.g., workstation 117 or monitor 118) to provide display of data, control, or other functions.

In some embodiments, device 10 may include an in-vivo video camera, for example, an imager, which may capture and transmit images of, for example, the GI tract while device 10 passes through the GI lumen. Other lumens and/or body cavities may be imaged and/or sensed by device 10. In some embodiments, the imager may include, for example, a Charge Coupled Device (CCD) camera or imager, a Complementary Metal Oxide Semiconductor (CMOS) camera or imager, a solid state camera or imager, a linear imaging sensor, a line imaging sensor, a full frame imaging sensor, a “camera on chip” imaging sensor, a digital camera, a stills camera, a video camera, or other suitable imagers, cameras, or image acquisition components.

In some embodiments, transmitter 141 of device 10 may include a wireless transmitter, e.g., able to operate using radio waves, able to transmit Radio Frequency (RF) signals, or able to transmit other types of wireless communication signals. For example, transmitter 141 may transmit wireless signals utilizing an antenna 21. In other embodiments, such as those where device 10 is or is included within an endoscope, transmitter 141 may transmit data via, for example, wire, cable, optical fiber and/or other suitable methods. Other known wired and/or wireless methods of transmission may be used.

In some embodiments, device 10 may optionally include a receiver 196, for example, a wired or wireless (e.g., RF) receiver, able to receive signals from an external transmitter. The received signals may include, for example, control signals or commands, e.g., to activate and/or otherwise control one or more components of device 10. Receiver may receive signals, e.g., from outside the patient's body, for example, through antenna 21 or through a different antenna or receiving element. In some embodiments, signals or data may be received by a separate receiving unit in device 10. In some embodiments, transmitter 141 and the receiver may optionally be implemented using a transceiver unit or an integrated transmitter-receiver unit.

Transmitter 141 may also include control capability, although control capability may be included in a separate component, e.g., a controller or processor. Transmitter 141 may include any suitable transmitter able to transmit image data, other sensed data, and/or other data (e.g., control data) to a receiving device. Transmitter 141 may also be capable of receiving signals/commands, for example from an external transceiver. For example, in some embodiments, transmitter 141 may include an ultra low power Radio Frequency (RF) high bandwidth transmitter, possibly provided in Chip Scale Package (CSP).

Power source 145 may include, for example, one or more batteries or power cells. For example, power source 145 may include silver oxide batteries, lithium batteries, other suitable electrochemical cells having a high energy density, or the like. Other suitable power sources may be used. For example, power source 145 may receive power or energy from an external power source (e.g., an electromagnetic field generator), which may be used to transmit power or energy to in-vivo device 10.

In some embodiments, power source 145 may be internal to device 10, and/or may not require coupling to an external power source, e.g., to receive power. Power source 145 may provide power to one or more components of device 10, for example, continuously, substantially continuously, or in a non-discrete manner or timing, or in a periodic manner, an intermittent manner, or an otherwise non-continuous manner. In some embodiments, power source 145 may provide power to one or more components of device 10, for example, not necessarily upon-demand, or not necessarily upon a triggering event or an external activation or external excitement.

In some embodiments, device 10 may include one or more illumination sources, for example one or more Light Emitting Diodes (LEDs), “white LEDs”, monochromatic LEDs, Organic LEDs (O-LEDs), thin-film LEDs, single-color LED(s), multi-color LED(s), LED(s) emitting viewable light, LED(s) emitting non-viewable light, LED(s) emitting Infra Red (IR) light, an emissive electroluminescent layer or component, Organic Electro-Luminescence (OEL) layer or component, or other suitable light sources.

Illumination sources may, for example, illuminate a body lumen or cavity being imaged and/or sensed. In some embodiments, illumination source(s) may illuminate continuously, or substantially continuously, for example, not necessarily upon-demand, or not necessarily upon a triggering event or an external activation or external excitement. In some embodiments, for example, illumination source(s) may illuminate a predefined number of times per second (e.g., two or four times), substantially continuously, e.g., for a time period of two hours, four hours, eight hours, or the like; or in a periodic manner, an intermittent manner, or an otherwise non-continuous manner.

In some embodiments, the components of device 10 may be enclosed within a housing or shell, e.g., capsule-shaped, oblong, oval, spherical, tubular, peanut-shaped, or having other suitable shapes and/or dimensions. The housing or shell may be substantially transparent or semi-transparent, and/or may include one or more portions, windows or domes (e.g., a dome-shaped window, or multiple dome-shaped windows) which may be substantially transparent or semi-transparent.

Data processor 114 may analyze the data received via external receiver/recorder 112 from device 10, and may be in communication with storage unit 119, e.g., transferring frame data to and from storage unit 119. Data processor 114 may provide the analyzed data to monitor 118, where a user (e.g., a physician) may view or otherwise use the data. In some embodiments, data processor 114 may be configured for real time processing and/or for post processing to be performed and/or viewed at a later time. In the case that control capability (e.g., delay, timing, etc) is external to device 10, a suitable external device (such as, for example, data processor 114 or external receiver/recorder 112 having a transmitter or transceiver) may transmit one or more control signals to device 10.

Monitor 118 may include, for example, one or more screens, monitors, or suitable display units. Monitor 118, for example, may display data sensed by sensing unit 500, one or more images and/or a stream of images captured and/or transmitted by device 10, e.g., images of the GI tract or of other imaged body lumen or cavity. Additionally or alternatively, monitor 118 may display, for example, control data, location or position data (e.g., data describing or indicating the location or the relative location of device 10), orientation data, and various other suitable data. In some embodiments, for example, sensed data, an image and its position (e.g., relative to the body lumen being sensed) or location may be presented using monitor 118 and/or may be stored using storage unit 119. Other systems and methods of storing and/or displaying collected image data and/or other data may be used.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An in-vivo device for in-vivo magnetic immunoassay analysis comprising:
a reaction channel, wherein the reaction channel is configured to accept an in-vivo fluid sample;
paramagnetic particles conjugated with a receptor specific to a target molecule;
a magnetic sensor to detect the paramagnetic particles bonded with the target molecule; and
a magnet.

2. The in-vivo device of claim 1 wherein the magnetic sensor is integrated onto a silicon chip.

3. The in-vivo device of claim 1 comprising
an external receiver for wirelessly receiving output from the in-vivo device, and
a processor for processing the output.

4. The in-vivo device of claim 1 wherein the magnetic sensor is a Hall Effect based sensor.

5. The in-vivo device of claim 1 comprising a pump, wherein the pump is to pump the in-vivo fluid sample into the reaction channel.

5. The in-vivo device of claim 1 comprising a pump, wherein the pump is to pump the in-vivo fluid sample into the reaction channel.

6. The in-vivo device of claim 5 wherein the pump is a piezoelectric pump.

7. The in-vivo device of claim 1 comprising a reagent reservoir, wherein the reagent reservoir stores a suspension of the paramagnetic particles.

8. The in-vivo device of claim 1 wherein the reaction channel includes a coating of the receptor specific to the target molecule.

9. The in-vivo device of claim 1 wherein the magnet is an electromagnet configured for inducing an alternating magnetic field.

10. The in-vivo device of claim 1 comprising a wireless transmitter to transmit an output from the magnetic sensor.

11. The in-vivo device of claim 1 comprising a waste chamber; to accept excess fluid sample from the reaction channel.

12. The in-vivo device of claim 1 wherein the in-vivo device is a swallowable capsule.

13. A method for in vivo magnetic immunoassay analysis, the method comprising:
collecting a fluid sample in-vivo;
introducing paramagnetic particles to the fluid sample in-vivo, wherein the paramagnetic particles are conjugated with a receptor specific to a target molecule;
applying a magnetic field in-vivo, in the vicinity of the paramagnetic particles; and
detecting in-vivo the paramagnetic particles that bonded to the target molecules.

14. The method of claim 13 comprising immobilizing the paramagnetic particles that bonded to the target molecules.

15. The method of claim 13 wherein the magnetic field is an alternating magnetic field.

16. The method of claim 13 comprising detecting changes in the magnetic field due to the paramagnetic particles bonding to a target molecule.

17. The method of claim 13 comprising flushing excess fluid sample to a waste chamber.

18. The method of claim 13 comprising transmitting by wireless connection, results from the detecting to an external source.

19. The method of claim 13 comprising inserting the in-vivo device into the GI tract.

20-25. (canceled)

26. The method of claim 18 comprising displaying the results to a user.
